(19) United States
(12) Patent Application Publication
(10) Pub. No.: US 2026/0130691 A1
(43) Pub. Date: May 14, 2026

(54) IRRIGATION FLUID BARRIER DEVICES AND ASSOCIATED SURGICAL SYSTEMS AND METHODS

(71) Applicant: Amplify Surgical, Inc., Irvine, CA (US)

(72) Inventors: Andy Wonyong Choi, Irvine, CA (US); Clark Hutton, Carlsbad, CA (US); Shigeru Tanaka, Half Moon Bay, CA (US); Martin Leugers, San Francisco, CA (US)

(21) Appl. No.: 19/384,617

(22) Filed: Nov. 10, 2025

Related U.S. Application Data
(60) Provisional application No. 63/719,049, filed on Nov. 11, 2024.
(60) Provisional application No. 63/779,765, filed on Mar. 28, 2025.

(51) Int. Cl. A61B 17/34; A61B 17/00; A61B 46/20

(52) U.S. Cl. CPC A61B 17/3423; A61B 46/20; A61B 2017/00477; A61B 2017/00951; A61B 2217/007

(57) ABSTRACT

Surgical systems and associated devices and methods are disclosed herein. In some embodiments, a surgical system includes an irrigation fluid barrier device positionable on a back of a patient. The irrigation fluid barrier device can include a core material and a covering enclosing the core material. The core material and the covering can be shaped to, when the device is positioned on the back of the patient, at least partially surround a port in the patient. The covering can be configured to direct irrigation fluid flowing out of the port in a direction away from the patient. The irrigation fluid barrier device can be U-shaped, rectangular-shaped, or ovoid-shaped, and/or can define a channel for the irrigation fluid to flow through, etc. The core material can include at least one of a gel or a foam. The covering can be configured to create a fluid-tight seal around the core material.

### CROSS-REFERENCE TO RELATED APPLICATIONS

The present application claims the benefit of U.S. Provisional Patent Application No. 63/719,049, filed Nov. 11, 2024, and U.S. Provisional Patent Application No. 63/779,765, filed Mar. 28, 2025, the disclosures of which are incorporated herein by reference in their entireties.

### TECHNICAL FIELD

The present technology relates generally to medical systems and, more particularly, to irrigation fluid barrier devices and associated surgical systems and methods.

### BACKGROUND

Spinal surgeries, such as laminectomy, discectomy, spinal fusion, vertebroplasty, kyphoplasty, foraminotomy, microdiscectomy, spinal tumor resection, and scoliosis correction, often require continuous irrigation to maintain a clear and sterile field. For example, such procedures may involve the removal of substantial soft tissue or bone, which generates blood, bone dust, tissue fragments, and other debris that must be cleared regularly to preserve visibility. Irrigation fluid can be introduced into a surgical site not only for maintaining visibility but also for cooling tissue and instruments, and for reducing the risk of infection by constantly flushing out contaminants. Without irrigation, the accumulation of debris and bodily fluids can obscure the surgical site and interfere with the medical procedure. However, introducing significant volumes of irrigation fluid into the confined space of a spinal surgical site can quickly lead to unintended fluid buildup. This accumulation often exceeds the capacity of traditional suction devices and absorbent materials, resulting in fluid overflow. Such overflow not only complicates the surgeon's work by obscuring the surgical field but also risks contamination of the surrounding area and prolongs the time needed for post-operative cleaning. Accordingly, there is a need for improved fluid management systems, devices, and/or related technologies.

### BRIEF DESCRIPTION OF THE DRAWINGS

Features, aspects, and advantages of the presently disclosed technology may be better understood with regard to the following drawings.

FIG. 1 is a side view of a multi-portal surgical system configured in accordance with embodiments of the present technology.

FIG. 2 is a partially schematic top view of a multi-portal surgical system including an irrigation fluid barrier device, each configured in accordance with embodiments of the present technology.

FIG. 3 is a partially schematic top view of a multi-portal surgical system including an irrigation fluid barrier device for directing flow of expelled irrigation fluid in accordance with embodiments of the present technology.

FIGS. 4A and 4B are partially schematic cross-sectional views of the multi-portal surgical system of FIG. 3 positioned on a patient.

FIGS. 5-10 are partially schematic top views of various irrigation fluid barrier devices, each configured in accordance with embodiments of the present technology.

FIG. 11 is an isometric view of an irrigation fluid barrier device configured in accordance with embodiments of the present technology.

FIGS. 12A and 12B are top and bottom views, respectively, of another irrigation fluid barrier device configured in accordance with embodiments of the present technology.

FIG. 13 is an isometric partially exploded view of another irrigation fluid barrier device configured in accordance with embodiments of the present technology.

FIG. 14 is an isometric, partially exploded view of the irrigation fluid barrier device of FIG. 13 in accordance with embodiments of the present technology.

FIG. 15 is a partially schematic top view of another multi-portal surgical system including irrigation fluid barrier devices for directing flow of expelled irrigation fluid in accordance with embodiments of the present technology.

FIG. 16 is a flowchart illustrating a method for irrigating a surgical site in a patient in accordance with embodiments of the present technology.

A person skilled in the relevant art will understand that the features shown in the drawings are for purposes of illustrations, and variations, including different and/or additional features and arrangements thereof, are possible.

### DETAILED DESCRIPTION

The following disclosure describes various embodiments of irrigation fluid barrier devices and associated systems and methods of use. An irrigation fluid barrier device can be positioned on a patient and shaped to direct irrigation fluid exiting a surgical site into a collection container, away from a physician's body/feet, or in another direction. More specifically, the irrigation fluid barrier device can be positioned and shaped to at least partially surround one or more ports in the patient through which the irrigation fluid can enter and exit the surgical site. For example, the irrigation fluid barrier device can partially or fully surround the ports and/or include a channel so that the irrigation fluid can be directed away from the patient in a specific direction (e.g., out of the open end of a U-shaped barrier, as viewed from above, through the channel). The irrigation fluid barrier device may be positioned and oriented to direct the irrigation fluid into a collection container, such as a bucket on the floor. In some embodiments, the irrigation fluid barrier device includes a core material (e.g., gel, foam) and a covering enclosing the core material. The core material can provide the structure and shape of the irrigation fluid barrier device, and the covering can form a fluid-tight seal around the core material to prevent the irrigation fluid from contacting the core material.

In some embodiments, the irrigation fluid barrier device can define a reservoir around the ports so that if the irrigation fluid flow rate exceeds the rate at which the device can direct the irrigation fluid away from the patient, the irrigation fluid can pool in the reservoir as opposed to, for example, overflowing and spilling in an undesired direction. In some embodiments, an incise drape can be positioned over the irrigation fluid barrier device so that once the incise drape adheres to the patient (e.g., the incise drape can include an adhesive layer that can stick to the patient's skin), the incise drape can fix or otherwise secure a position of the irrigation fluid barrier device relative to the patient. In some embodiments, the irrigation fluid barrier device is connected to, or part of, the incise drape such that the integrated irrigation fluid barrier device is held in place by the incise drape adhered to the patient. In some embodiments, the irrigation fluid barrier device includes an adhesive (e.g., a pressure-sensitive adhesive, an antibacterial adhesive, etc.) that can be adhered to a drape, patient skin, or other covering at the surgical site. This allows the irrigation fluid barrier device to be attached to different locations during the surgery. A user can reposition the irrigation fluid barrier device any number of times during the procedure for flexibility. The irrigation fluid barrier device can also be used as a surgical pillow that supports, for example, a surgeon or other user holding a surgical instrument (e.g., an endoscope) in the patient. The reservoir can be uncovered to allow access to the ports.

In some embodiments, the irrigation fluid barrier device includes an elongate irrigation member (“elongate member”) and one or more support bases temporarily or permanently coupleable to the elongate member. The elongate member can serve as the barrier or reservoir-forming portion of the irrigation fluid barrier device. The support bases can be shaped and sized to removably attach to the elongate member, and can also interface with the patient underneath. In particular, the support bases can have various shapes and can be attached to various portions of the elongate member. Accordingly, the irrigation fluid barrier device can be a modular device in which the elongate member can be used with various combinations of support bases. A surgeon or other user can select the appropriate support bases and the appropriate placements thereof to secure the elongate member on the patient in the desired shape while conforming to the shape of the specific patient.

In the Figures, identical reference numbers identify generally similar, and/or identical, elements. Many of the details, dimensions, and other features shown in the Figures are merely illustrative of particular embodiments of the disclosed technology. Accordingly, other embodiments can have other details, dimensions, and features without departing from the spirit or scope of the disclosure. In addition, those of ordinary skill in the art will appreciate that further embodiments of the various disclosed technologies can be practiced without several of the details described below.

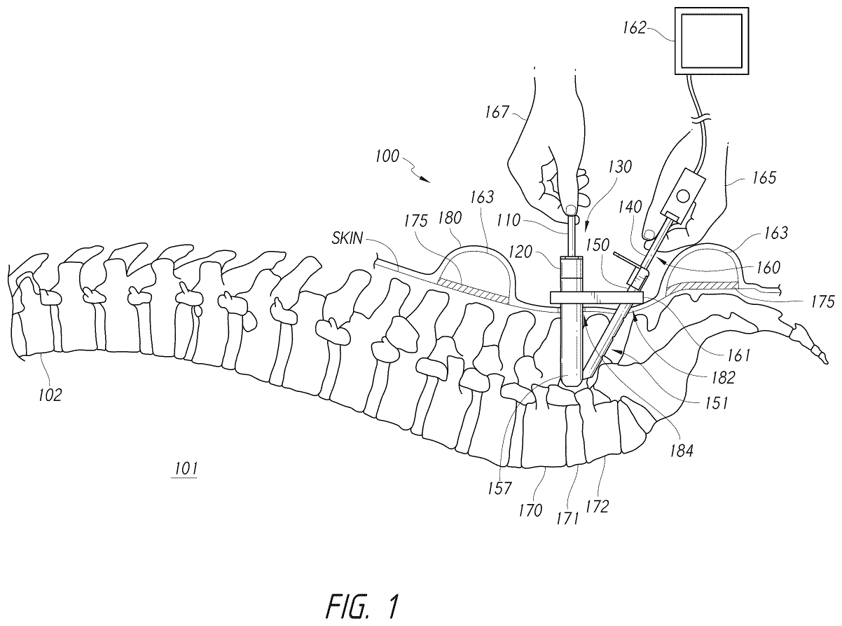

FIG. 1 is a side view of a multi-portal surgical system 100 (“the system 100”) positioned along patient 101 and configured in accordance with embodiments of the present technology. The system 100 can include an instrument assembly 130, an instrument assembly 160, an irrigation fluid barrier device 163, and a drape 180. The instrument assembly 130 can include an instrument 110 and an optional cannula 120. The instrument assembly 160 can include an instrument 140 and an optional cannula 150 (e.g., a split cannula). The instrument 110 can be used to remove tissue (e.g., an intervertebral disc 171 between adjacent vertebrae 170, 172, tissue contributing to stenosis, etc.), form access paths to implantation sites, prepare an implantation site by, for example, moving organs or tissue (e.g., moving nerve tissue), prepare vertebral bodies (e.g., roughening or shaping vertebral endplates), and/or the like. The instrument 110 and/or the cannula 120 can be delivered through a first port or incision 184, and may be secured by an instrument holder 161.

The instrument 140 can be used to endoscopically view inside the patient while providing irrigation. The instrument 140 and the cannula 150 can be delivered through a second port or incision 182, and may be secured by the instrument holder 161. The irrigation fluid can be pressurized inside the patient and can be expelled out of one or both of the ports 182, 184. The irrigation fluid barrier device 163 can be configured to hold or contain the expelled irrigation fluid to prevent, inhibit, or limit flow of the irrigation fluid onto the floor. In some embodiments, the irrigation fluid barrier device 163 has a flexible reservoir body configured with sufficient rigidity to prevent collapse of the flexible reservoir body when the reservoir is at least partially filled with saline. In some embodiments, the irrigation fluid barrier device 163 can direct the irrigation fluid into a collection container, such as a bucket or a bin, to prevent the irrigation fluid from flowing onto the surgical room floor. This can help maintain a dry surgical floor to inhibit or prevent slipping on the surgical floor or other adverse events.

The instrument 140 can include a lumen through which irrigation fluid flows to irrigate the surgical site. For example, saline, or another suitable liquid, can be pumped through the instrument 140 to remove tissue (e.g., loose tissue, bone dust, etc.) or other material impairing visualization. The instrument 140 can also include one or more lumens (e.g., irrigation return lumens, vacuum lumens, etc.) through which the irrigation liquid can be withdrawn. In some embodiments, the irrigation fluid exits the surgical site via the port 184 of the patient 101.

The instrument 140 can be inserted directly through the port 182 without the use of a cannula. In some embodiments, the cannula 150 can be positioned in the incision 182 for facilitating insertion or removal of the instrument 140. The instrument 140 can be moved distally and/or laterally out of the cannula 150, which can be positioned in a first incision or port 182 in the patient 101, to access a relatively large working space along a spine 102 of the patient 101. The split cannula 150 can have an open front side 151 (illustrated facing the inferior direction relative to the patient) to allow a distal portion of the instrument 140 to be moved laterally into and out of one side of the cannula 150. Irrigation techniques, visualization devices, instruments, cannulas, and visualization and surgical techniques are discussed in U.S. application Ser. No. 17/902,685 and U.S. Pat. No. 11,678,906, which are incorporated by reference in their entireties.

In some embodiments, the instrument assembly 160 can include a visualization assembly that provides intraoperative endoscopic viewing of workspaces, delivery paths, organs, tissue (e.g., nerve tissue) implantation sites, implants, interbody fusion devices (e.g., before, during, and/or after delivery), instrument(s) (including dispensers, dilators, decompression instruments, etc.), and other areas or features of interest. For example, the instrument 140 can include a visualization instrument having a low-profile fiber-optic endoscope positioned directly through the first port 182. The instrument 140 can include one or more endoscopes having, without limitation, fiber optics (e.g., optical fibers), lenses, imaging devices, working lumens, light source controls, or the like for direct viewing or viewing via a display 162 (e.g., an electronic screen, a monitor, etc.). The instrument 140 can illuminate the body cavity and enable high-resolution video visualization. A light source (e.g., a laser, light-emitting diode, etc.) located near or at the proximal end of the fiber optics can be used to transmit light to the distal end and provide illuminating light. This enables a surgeon to safely navigate into the subject's body and to illuminate specific body anatomy to view vertebral spacing, vertebral structures, nerves, bony buildup (e.g., buildup that could be irritating and pressing against nerves contributing to nerve compression), etc. In some embodiments, visualization optics for vision and illumination are included within the distal tip of the instrument 140. The configuration and functionality of the instrument 140 can be selected based on the desired field of view, viewing resolution, pan/zoom functionality, or the like.

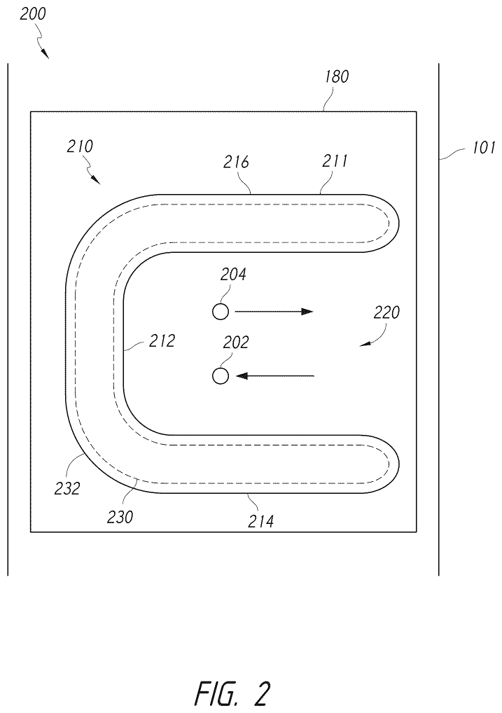

The irrigation fluid barrier device 163 can be positionable on the patient 101 (e.g., on the patient's skin). As discussed in further detail herein with reference to FIGS. 2-10, the irrigation fluid barrier device 163 can be shaped to at least partially surround the first port 182 and/or the second port 184. In some embodiments, the irrigation fluid barrier device 163 is coupled directly or indirectly to the patient's skin to prevent irrigation fluid from escaping or flowing underneath the irrigation fluid barrier device 163. For example, an adhesive surface or layer 175 can be attached to the irrigation fluid barrier device 163 and can be configured to form a fluid-tight seal with the drape 180 applied to the patient, with the patient's skin, and/or the like. The irrigation fluid barrier device 163 can have a generally flat bottom surface such that the adhesive layer 175 can be positioned below the irrigation fluid barrier device 163 to adhere to the patient's skin, as shown. In other embodiments, the adhesive layer 175 can be positioned elsewhere such as above the irrigation fluid barrier device 163 to adhere to an underside of the drape 180. In operation, as irrigation fluid is delivered to the surgical site (e.g., by the instrument 140 and via the first port 182) and exits the surgical site (e.g., via the second port 184), the irrigation fluid barrier device 163 can block or limit flow in one or more directions, collect irrigation fluid, and/or direct the irrigation fluid in a desired direction along and/or away from the patient 101.

In some embodiments, the irrigation fluid barrier device 163 can also serve as an ergonomic pillow and be used to position a user's body during the surgical procedure. In the illustrated embodiment, the irrigation fluid barrier device 163 is configured to support the user's hand 167 holding the instrument assembly 160. The irrigation fluid barrier device 163 can support the user alone or in conjunction with any number of pillows. Additionally, different irrigation fluid barrier devices 163 can be used for corresponding sets for surgical actions. For example, a first irrigation fluid barrier device can be used to perform a first decompression procedure at a first level of the patient's spine 102, a second irrigation fluid barrier device can be used to perform a second decompression procedure at a second level of the patient's spine 102, and a third irrigation fluid barrier device can be used to perform one or more implantation procedures at the first and/or second level or other levels.

The drape 180 can be positioned over the irrigation fluid barrier device 163 and the patient 101. The drape can include an iodine-impregnated material, which provides an additional antimicrobial barrier to help reduce the risk of infection at the surgical site. In some embodiments, the drape 180 includes an incise drape with an adhesive layer so that the drape 180 can stick to the irrigation fluid barrier device 163 and/or the skin of the patient 101. The adhesive layer can include, without limitation, pressure-sensitive adhesives, acrylic adhesives, iodophor adhesives, ultraviolet (UV) or light-curing adhesives, and/or the like. The drape 180 can thereby fix or otherwise secure a position of the irrigation fluid barrier device 163 relative to the patient 101. The drape 180 can include one or more fenestrations (e.g., holes or openings) aligned with the first port 182 and/or the second port 184.

In some embodiments, both the irrigation fluid barrier device 163 and the drape 180 can include adhesive surfaces for attaching to the patient's skin or each other. In the illustrated embodiment, the drape 180 is placed over the irrigation fluid barrier device 163 and the patient's skin, and the adhesive layer of the drape 180 and/or the adhesive layer 175 can keep the irrigation fluid barrier device 163 at a secure position on the patient. In some embodiments, the drape 180 can be placed directly against the patient's skin, and the irrigation fluid barrier device 163 can be positioned on an upper surface of the drape 180. A lower surface of the irrigation fluid barrier device 163 can be configured to adhere directly to the drape 180. For example, a lower surface of the drape 180 can have an adhesive surface for adhering to the patient's skin, and the adhesive layer 175 can keep the irrigation fluid barrier device 163 attached to an upper surface of the drape 180. In embodiments of the present technology, the adhesive layer 175, the adhesive layer of the drape 180, and/or the irrigation fluid barrier device 163 can be configured to provide a seal (a fluid-tight seal, an airtight seal, etc.) to inhibit or prevent irrigation fluid from flowing between the irrigation fluid barrier device 163 and the drape 180, beyond a reservoir defined by the irrigation fluid barrier device 163, and/or the like. In some procedures, another drape can be positioned above and overlay the irrigation fluid barrier device 163. The number, configuration, and features of the drapes can be selected based on the procedure. Thus, the instrument 140 can be inserted and deliver irrigation fluid through the first port 182 and the corresponding fenestration of the drape 180, and the irrigation fluid can exit the surgical site via the first port 182 and/or the second port 184 and the corresponding fenestration of the drape 180.

FIG. 2 is a partially schematic top view of a multi-portal surgical system 200 (“the system 200”) including an irrigation fluid barrier device 210, each configured in accordance with embodiments of the present technology. The system 200 can be an example of the system 100 of FIG. 1, and the irrigation fluid barrier device 210 can be an example of the irrigation fluid barrier device 163 of FIG. 1. As shown, the system 200 can also include the drape 180 positioned over the irrigation fluid barrier device 210, both positioned on the patient 101.

In the illustrated embodiment, the irrigation fluid barrier device 210 is U-shaped. More specifically, the irrigation fluid barrier device 210 can include a reservoir body or wall 211 partially surrounding the ports 202, 204. The wall 211 can include a first segment 212 extending along a first direction, a second segment 214 extending from one end of the first segment 212 along a second direction substantially perpendicular to the first direction, and a third segment 216 extending from the other end of the first segment 212 along the second direction. Each of the first segment 212, the second segment 214, and the third segment 216 can have a length between 5 and 30 inches, between 10 and 20 inches (e.g., 17 inches), or other length suitable for extending at least partially across or along the back of a patient. The wall 211 and the patient's body form a reservoir 220 (e.g., an open reservoir), and the U-shaped irrigation fluid barrier device 210 includes an elongate body having a first end and an opposing second end such that the gap therebetween defines an exit opening of the reservoir 220. The irrigation fluid barrier device 210 can be oriented by a physician such that the exit opening (e.g., the open side of the reservoir 220) faces a side of the patient 101.

In some embodiments, the irrigation fluid barrier device 210 includes a core material 230 and a covering 232 enclosing the core material 230. The core material 230 can include a gel, a foam, and/or other suitable material, and can provide the structure and shape (e.g., the illustrated U shape) of the irrigation fluid barrier device 210. In some embodiments, the core material 230 can include a moldable region and/or a non-formable support region or portion. The moldable region can include, for example, one or more thermoformable materials, thermosetting materials, curable materials, foams (e.g., closed cell foam, open cell foam, etc.), inflatable members, and/or the like. The moldable region can include a material that is more compressible than the material of the support portion. This allows for local deformation to enhance comfort of the user while the support portion provides stability relative to the underlying patient. The moldable region can have a convex region along which the user's hand or wrist can roll. The moldable region can have a concave region for holding the user's hand or wrist generally stationary. For example, the concave region can receive the backside of the user's hand or wrist. The moldable region can be configured to be molded to the user's body such that the irrigation fluid barrier device 210 comprises a user-specific, disposable surgical support pillow.

The covering 232 can include vinyl, polyvinyl chloride (PVC), silicone, and/or other suitable material, and can create a fluid-tight seal around the core material 230. Thus, the covering 232 can protect the core material 230 from any fluids or contaminants that may damage the core material 230. The covering 232 can also be flexible and/or moldable to form around the shape of the core material 230, enabling the core material 230 to define the geometry of the irrigation fluid barrier device 210.

The patient 101 can have a first incision or port 202 and a second incision or port 204, which can be examples of the first port 182 and the second port 184, respectively, of FIG. 1. The first port 202 and the second port 204 can be spaced apart (e.g., along the length of the patient's spine) and can be in fluid communication with one another inside the patient 101 (e.g., via the surgical site). Instruments, such as the instrument 140 and the instrument 110 of FIG. 1, which may be inserted in the first and second ports 202, 204, respectively, are omitted in FIG. 2. As shown, the irrigation fluid barrier device 210 can be positioned and sized to partially surround the first port 202 and the second port 204 such that the reservoir 220 is in fluid communication with the surgical site via the first port 202 and/or the second port 204.

The drape 180 can be positioned over the irrigation fluid barrier device 210 and the patient 101, and can include fenestrations (e.g., holes or openings) aligned with the first port 202 and/or the second port 204. In some embodiments, the drape 180 includes an adhesive layer so that the drape 180 can stick to the irrigation fluid barrier device 210 and/or the skin of the patient 101. The drape 180 can thereby fix or otherwise secure a position of the irrigation fluid barrier device 210 relative to the patient 101. The drape 180 can be flexible such that the drape 180 can form around the irrigation fluid barrier device 210, enabling the irrigation fluid barrier device 210 to define the shape of the reservoir 220 above the drape 180.

In operation, an instrument (e.g., the instrument 140 of FIG. 1) can be positioned at least partially in the first port 202 (and through a corresponding fenestration of the drape 180) and operated to deliver irrigation fluid (e.g., saline) therethrough. The physician can hold the instrument with one hand while using the irrigation fluid barrier device 210 as a support pillow (e.g., supporting the physician's hand and/or wrist), as generally illustrated in FIG. 1. The irrigation fluid can travel into the surgical site via the first port 202 and out through the second port 204 (and through a corresponding fenestration of the drape 180), carrying materials (e.g., bone, tissue, blood, and/or other debris) out of the surgical site. The irrigation fluid and the materials can then at least partially pool in the reservoir 220 (e.g., on the drape), and the U shape of the irrigation fluid barrier device 210 can direct the irrigation fluid and the materials along and/or away from the patient 101, such as toward and beyond a side of the patient 101. Therefore, the irrigation fluid barrier device 210 can, during a medical procedure, help manage the irrigation fluid, which may be delivered to and exit the surgical site at a relatively high flow rate, and/or serve as a support pillow for the physician.

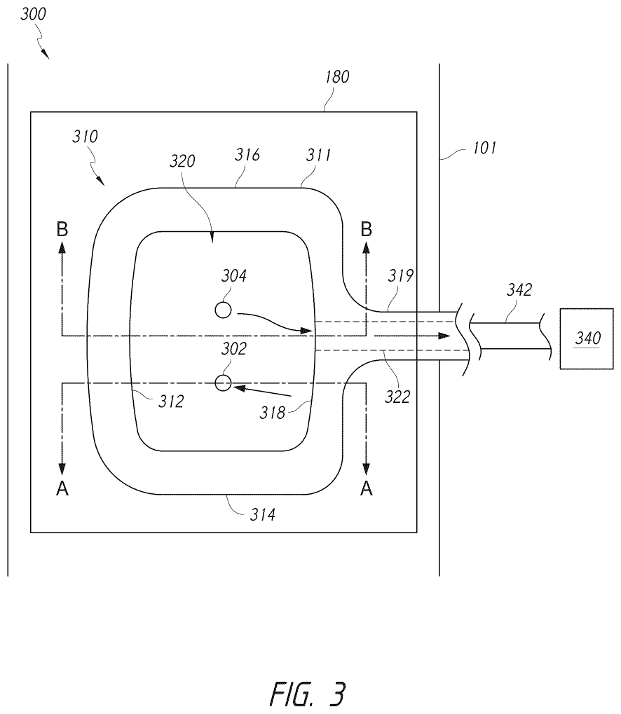

FIG. 3 is a partially schematic top view of a multi-portal surgical system 300 (“the system 300”) including an irrigation fluid barrier device 310, each configured in accordance with embodiments of the present technology. The system 300 can be an example of the system 100 of FIG. 1, and the irrigation fluid barrier device 310 can be an example of the irrigation fluid barrier device 163 of FIG. 1. As shown, the system 300 can also include the drape 180 positioned over the irrigation fluid barrier device 310, both positioned on the patient 101, and a container or collector 340 positioned away from the patient 101.

In the illustrated embodiment, the irrigation fluid barrier device 310 has a generally rectangular shape, as viewed from above. More specifically, the irrigation fluid barrier device 310 can include a reservoir body or wall 311 surrounding the ports 302, 304 and an out-flow conduit 319. The wall 311 and the patient's body form a reservoir 320 for collecting the irrigation fluid. The out-flow conduit 319 is connected to the wall 311 and includes a channel or passageway 322 (e.g., an open channel, a closed channel) to allow the irrigation fluid to flow out of the reservoir 320 and into the container 340. In some embodiments, the system 300 further includes a flow tube 342 that can be coupled to the irrigation fluid barrier device 310 (e.g., to the passageway 322) to further direct the irrigation fluid away from the patient and toward the container 340. The container 340 can be emptied or replaced any number of times during a surgical procedure.

The wall 311 can include a first segment 312 extending along a first direction, a second segment 314 extending from one end of the first segment 312 along a second direction substantially perpendicular to the first direction, a third segment 316 extending from the other end of the first segment 312 along the second direction, and a fourth segment 318 extending between the second segment 314 and the third segment 316 along the first direction. Each of the first segment 312, the second segment 314, the third segment 316, and the fourth segment 318 can have a length between 5 and 30 inches, between 10 and 20 inches (e.g., 17 inches), or other length suitable for extending at least partially across or along the back of a patient. The wall 311 can define the reservoir 320 (e.g., an at least partially enclosed reservoir).

Moreover, the out-flow conduit 319 extends from the wall 311 and beyond the side of the patient 101. The out-flow conduit 319 can be attached to or formed integrally with the fourth segment 318 and/or other parts of the irrigation fluid barrier device 310. The passageway 322 can be in fluid communication with the reservoir 320 and can be open at a distal end of the out-flow conduit 319. In some embodiments, one or more fluidic devices can be positioned along the passageway 322. For example, the fluidic devices can include one or more valves that can be moved from an open configuration to a closed configuration to inhibit or prevent fluid flow. A user can then empty the container 340 without fluid flowing onto the floor. Once the container 340 is replaced, the one or more valves can be moved to the open configuration to allow irrigation fluid to flow along the passageway 322 and into the container 340.

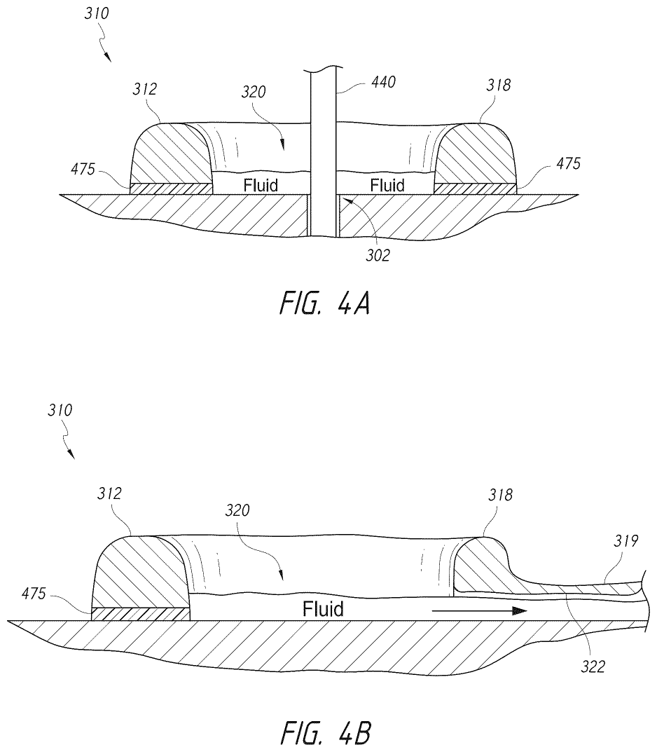

FIGS. 4A and 4B are partially schematic cross-sectional views of the system 300 along planes A-A and B-B, respectively, of FIG. 3. The drape 180 is omitted for illustrative purposes. As shown in FIG. 3, plane A-A extends through the first port 302 and plane B-B extends through the passageway 322. Referring first to FIG. 4A, an instrument 440 (not shown in FIG. 3) is shown at least partially inserted in the first port 302. The instrument 440 can be an example of the instrument 140 of FIG. 1, and can be configured to deliver irrigation fluid. Thus, the irrigation fluid can be delivered through the first port 302, and can exit via the first port 302 and/or the second port 304 (not shown in FIG. 4A). An adhesive layer 475 (e.g., the adhesive layer 175 of FIG. 1) can be positioned to attach the irrigation fluid barrier device 310 to the patient's skin. As shown, the irrigation fluid barrier device 310 can have a substantially flat bottom surface. In other embodiments, however, the irrigation fluid barrier device 310 is attached to the patient via the drape, etc. The adhesive layer 475, the drape, and/or the irrigation fluid barrier device 310 can form a fluid-tight seal with the patient's skin. Therefore, as the irrigation fluid exits the working space in the patient via the first port 302 and/or the second port 304, the irrigation fluid can pool in the reservoir 320 and around the instrument 440.

Referring next to FIG. 4B, the passageway 322 of the out-flow conduit 319 is in fluid communication with the reservoir 320 such that the irrigation fluid pooled in the reservoir 320 can flow out and away from the patient through the passageway 322. The drape (not shown in FIGS. 4A and 4B) can include one or more additional fenestrations to allow the irrigation fluid to flow into the passageway 322. In the illustrated embodiment, the out-flow conduit 319 does not include material at its underside and the passageway 322 is an open channel. Thus, the out-flow conduit 319 and the patient's skin co-define the passageway 322 through which the irrigation fluid can flown. Therefore, as shown, the irrigation fluid flowing through the passageway 322 travels over the skin of the patient (and/or the drape). In other embodiments, however, the out-flow conduit 319 can include material at its underside such that the passageway 322 is a closed channel.

Referring to FIGS. 3-4B together, in operation, the instrument 440 can be operated to deliver irrigation fluid (e.g., saline) to the surgical site via the first port 302. The physician can hold the instrument 440 with one hand while using the irrigation fluid barrier device 310 as a support pillow (e.g., supporting the physician's hand and/or wrist), as generally illustrated in FIG. 1. The irrigation fluid can travel through the surgical site, collect debris, and exit via the second port 304 (FIG. 3). The irrigation fluid and the debris can then pool in the reservoir 320. In some embodiments, the irrigation fluid barrier device 310 is positioned such that the entry of the passageway 322 is at the lumbar curve of the patient, and there is minimal pooling in the reservoir 320 (e.g., the irrigation fluid travels directly from the second port 304 to the passageway 322). The irrigation fluid and the debris traveling through the passageway 322 can then exit the out-flow conduit 319 and into, e.g., the container 340 (e.g., a bucket, etc.). Therefore, the irrigation fluid barrier device 310 can, during a medical procedure, help manage the irrigation fluid, which may be delivered to and exit the surgical site at a relatively high flow rate, and/or serve as a support pillow for the physician.

In some embodiments, the irrigation fluid barrier device 310 can be positioned and/or oriented differently from the illustrated embodiment of FIG. 3. For example, the out-flow conduit 319 can be positioned to laterally align with the first port 302 or the second port 304 (e.g., as opposed to neither as shown). In another example, the out-flow conduit 319 can be oriented to extend in a perpendicular direction (e.g., along the back of the patient 101). Also, while FIGS. 4A and 4B illustrate the cross-section of each of the first segment 312 and the fourth segment 318 as generally dome-shaped, it is appreciated that the irrigation fluid barrier device 310 of other embodiments can have other cross-sectional shapes. Furthermore, while not illustrated in FIGS. 3-4B, the irrigation fluid barrier device 310 can include a core material (e.g., the core material 230 of FIG. 2) and a covering (e.g., the covering 232), and the core material can define the shape of the irrigation fluid barrier device 310 and can include the first segment 312, the second segment 314, the third segment 316, the fourth segment 318, the out-flow conduit 319, etc.

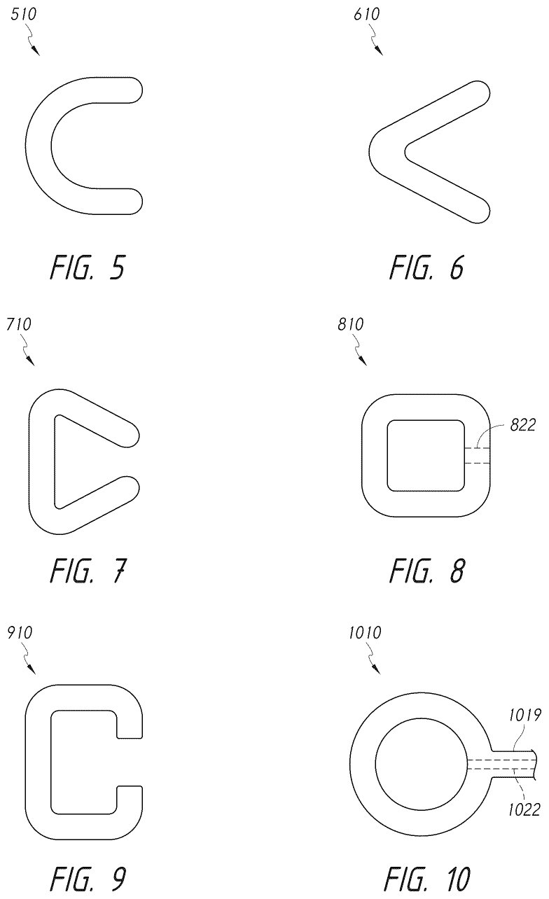

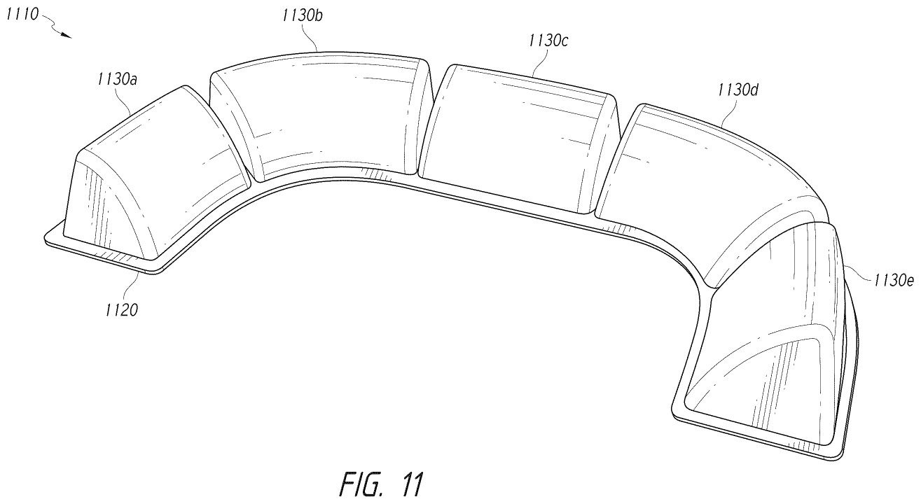

FIGS. 5-10 are partially schematic top views of various irrigation fluid barrier devices, each configured in accordance with embodiments of the present technology. FIG. 5 illustrates an irrigation fluid barrier device 510 having a semicircular shape such that irrigation fluid can be directed out the open end of the semicircle. FIG. 6 illustrates an irrigation fluid barrier device 610 having a V shape such that irrigation fluid can be directed out the open end of the V shape. FIG. 7 illustrates an irrigation fluid barrier device 710 having an open triangular shape such that irrigation fluid can be directed out the open vertex of the open triangular shape. FIG. 8 illustrates an irrigation fluid barrier device 810 having a rectangular or square shape and a channel 822 such that irrigation fluid can be directed out through the channel 822. In some embodiments, the irrigation fluid barrier device 810 omits the channel, and the irrigation fluid pooled in the reservoir defined by the closed rectangular shape can be removed using a suction, pump, or other device positioned in the reservoir. FIG. 9 illustrates an irrigation fluid barrier device 910 having a rectangular or square shape with a gap on one side such that irrigation fluid can be directed out through the gap. FIG. 10 illustrates an irrigation fluid barrier device 1010 having a circular or ovoid shape and an extension segment 1019 extending therefrom and defining a channel 1022 therein such that irrigation fluid can be directed out through the channel 1022.

FIG. 11 is an isometric view of an irrigation fluid barrier device 1110 configured in accordance with embodiments of the present technology. The irrigation fluid barrier device 1110 can include a base 1120 and a plurality of pillow segments (individually labeled 1130a-1130e, collectively referred to as “the pillow segments 1130”). The base 1120 can include one or more layers (e.g., films) that can rest against the patient's skin. For example, the base 1120 can be composed of silicone, plastic (e.g., polyurethane, polyethylene, polytetrafluoroethylene (PTFE)), and/or the like. In some embodiments, the base 1120 includes an adhesive layer that releasably secures the irrigation fluid barrier device 1110 to the patient's skin during a procedure.

The pillow segments 1130 can be coupled to the base 1120 in an arrangement that defines gaps between adjacent ones of the pillow segments 1130. In some embodiments, the pillow segments 1130 are separated from one another and are held together by the base 1120. In some embodiments, the pillow segments 1130 are integrally formed and connected to one another (e.g., near the base 1120). In the illustrated embodiment, the base 1120 is generally C-shaped and the pillow segments 1130 are arranged in a corresponding C-shaped pattern. As shown, each of the pillow segments 1130 can be wedge-shaped such that its sloped surface faces the inside of the C-shape. Each of the pillow segments 1130 can include a core material (e.g., gel, foam) and a covering enclosing the core material. The core material can provide the structure and shape of the pillow segments 1130, and the covering can form a fluid-tight seal around the core material to prevent the irrigation fluid from contacting the core material.

In operation, the irrigation fluid barrier device 1110 can comfortably support one or more body parts of a physician or other user, such as a hand, a wrist, a forearm, and/or the like. The fluid-tight contact between the base 1120 and the patient's skin and/or the shape and height of the pillow segments 1130 can direct irrigation fluid in a desired direction, as generally discussed above. In some embodiments, having separated pillow segments 1130 allows the irrigation fluid barrier device 1110 to be folded and thus stored and/or transported with a smaller profile. In some embodiments, the gaps between the pillow segments 1130 can receive one or more surgical tools, allowing the physician to easily and readily switch between different tools.

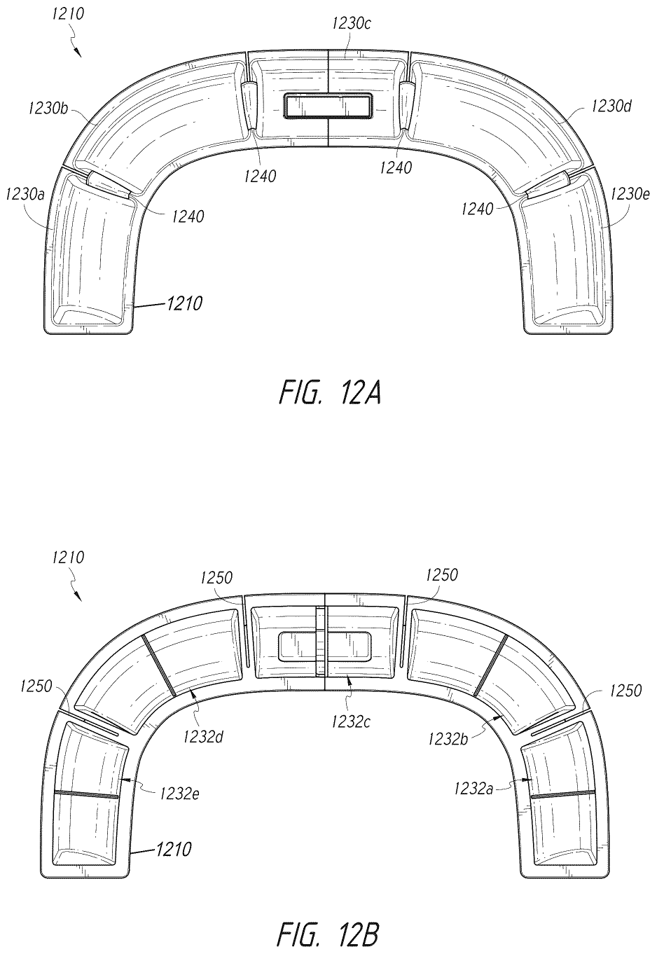

FIGS. 12A and 12B are top and bottom views, respectively, of another irrigation fluid barrier device 1210 configured in accordance with embodiments of the present technology. The irrigation fluid barrier device 1210 can be generally similar to the irrigation fluid barrier device 1110 of FIG. 11. For example, referring first to FIG. 12A, the irrigation fluid barrier device 1210 can include a base 1220 and a plurality of pillow segments (individually labeled 1230a-1230e, collectively referred to as “the pillow segments 1230”). Unlike the illustrated embodiment of FIG. 11, however, the irrigation fluid barrier device 1210 can further include a plurality of webbings 1240, each extending between adjacent ones of the pillow segments 1230. Each of the webbings 1240 can also extend from the base 1220 to a desired height such that the webbings 1240 can prevent fluid flow in between adjacent ones of the pillow segments 1230. The plurality of webbings 1240 can include a plurality of thin membranes or films, as shown, or thicker components that occupy more of the volume between adjacent ones of the pillow segments 1230. The plurality of webbings 1240 can be composed of an elastic material, a non-elastic material, or other suitable material.

Referring next to FIG. 12B, the pillow segments 1230 can define corresponding cavities 1232a-1232e extending from the bottom side of the base 1220. In other embodiments, however, the base 1220 can extend underneath each of the pillow segments 1230 such that the pillow segments 1230 are solid (e.g., full) or hollow. As shown, the base 1220 can also include a plurality of slots 1250 between adjacent ones of the pillow segments 1230, each extending radially inward from a radially outward side of the base 1220. The slots 1250 can facilitate reshaping, folding, storage, and/or the like of the irrigation fluid barrier device 1210. In some embodiments, the irrigation fluid barrier device 1210 is integrally formed and/or does not require a sheet (e.g., an iodine sheet).

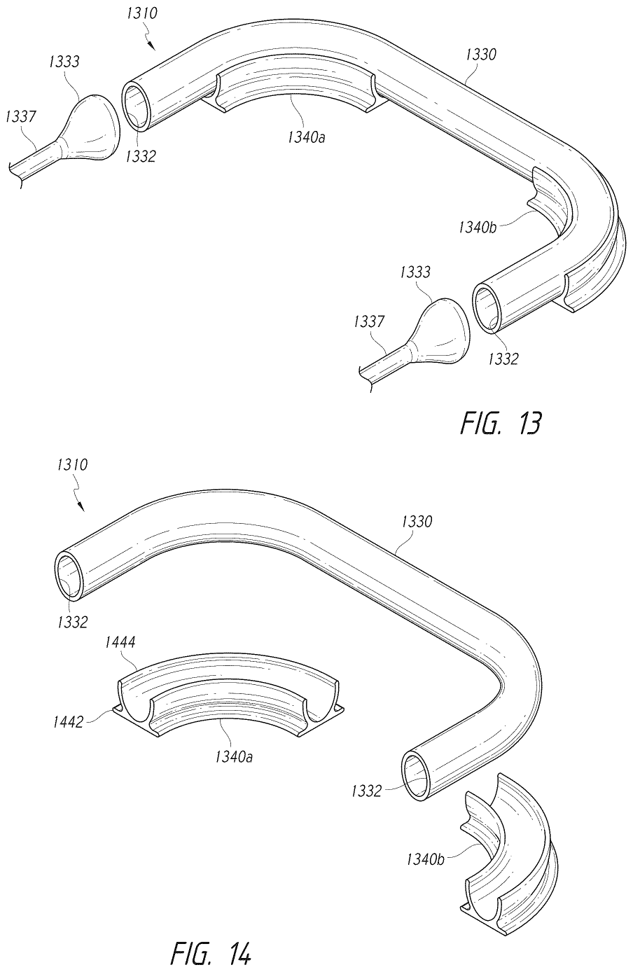

FIG. 13 is an isometric view of another irrigation fluid barrier device 1310 in an assembled state and configured in accordance with embodiments of the present technology. FIG. 14 is an isometric, partially exploded view of the irrigation fluid barrier device 1310. Referring to FIGS. 13 and 14 together, the irrigation fluid barrier device 1310 can include an elongate member 1330 and one or more support bases (individually labeled 1340a and 1340b, collectively referred to as “the support bases 1340”). As discussed in greater detail below, each support base 1340 can be removably coupled to a portion of the elongate member 1330 via adhesives, snap-fit, friction-fit, fasteners, and/or other suitable coupling mechanisms.

The elongate member 1330 can be a tube that is flexible or rigid (e.g., pre-formed) such that the elongate member 1330 can have C-curve shape, as shown in FIGS. 13 and 14, or other shape (e.g., as shown in FIGS. 5-10). The elongate member 1330 can be made of silicone, plastic (e.g., polyurethane, polyethylene, polytetrafluoroethylene (PTFE)), and/or other suitable material. The inside of the elongate member 1330 can be filled with fluid (e.g., air, water), a gel material, a foam material, a solid rod, etc. In some embodiments, the elongate member 1330 is an inflatable structure that can be substantially flattened and/or rolled-up, then filled with air or other fluid prior to use. In the illustrated embodiment, the elongate member 1330 includes two open ends 1332 on either end and has a circular cross-section. In other embodiments, however, the elongate member 1330 can have sealed or sealable ends on either or both ends, and/or a different cross-sectional shape such as elliptical/oval, triangular, rectangular, etc. In some embodiments, the irrigation fluid barrier device 1310 further includes connectors 1333 (not shown in FIG. 14) coupleable to the open ends 1332. The connectors 1333 can be coupled to conduits 1337 (e.g., hoses, fluid lines, etc.).

The support bases 1340 can be made of silicone, plastic (e.g., polyurethane, polyethylene, polytetrafluoroethylene (PTFE)), and/or other suitable material. As shown in FIG. 14, each support base 1340 can have a patient-interface portion 1442 and a barrier-interface portion 1444. The patient-interface portion 1442 can include a generally flat base, flanges and/or otherwise have a form factor that is substantially flat (as shown), curved, and/or deformable to interface a patient positioned underneath the irrigation fluid barrier device 1310. For example, the patient-interface portion 1442 can be adhered to the patient's skin, removably attached thereto via adhesive tapes, and/or the like. The barrier-interface portion 1444 can be shaped and sized to receive a portion of the elongate member 1330. In the illustrated embodiment, for example, the barrier-interface portion 1444 defines an open channel (e.g., U shaped channel, semi-circular channel, etc.) that is curved by 90°. The open channel can have a shape corresponding to the cross-sectional shape of the elongate member 1330. In other embodiments, the barrier-interface portion 1444 can be substantially straight or curved by a different angle (e.g., between 30-179°). The support bases 1340 can include one or more straps, ties, hook and loop fasteners, and/or the like configured to hold or couple to the elongate member 1330.

Referring to FIGS. 13 and 14 together, the irrigation fluid barrier device 1310 is a modular irrigation fluid barrier device that can be customized to the patient and/or the specific needs of the medical procedure. As previously mentioned, the elongate member 1330 can engage and be coupled to each support base 1340 via adhesives, snap-fit, friction-fit, fasteners, and/or other suitable coupling mechanisms. Different combinations of the elongate member 1330 and the support bases 1340 can be used to meet specific needs. For example, instead of using two curved support bases 1340 (as illustrated), the operator may opt to use three straight support bases 1340 at different portions of the elongate member 1330 and thereby maintain the C-shape of the elongate member 1330. Moreover, if the elongate member 1330 is flexible, the operator can bend or otherwise deform the elongate member 1330 to better conform to the patient's geometry, modify the shape of the elongate member 1330 mid-procedure, etc. Accordingly, the operator (or other user) can select the appropriate support bases 1340 and the appropriate placements thereof to secure the elongate member 1330 on the patient in the desired shape while conforming to the shape of the specific patient.

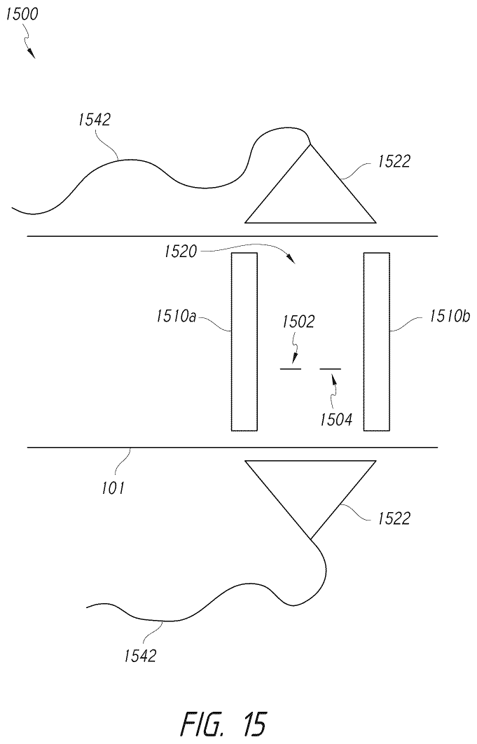

FIG. 15 is a partially schematic top view of another multi-portal surgical system 1500 (“the system 1500”) configured in accordance with embodiments of the present technology. The system 1500 can include one or more irrigation fluid barrier devices (individually labeled 1510a and 1510 b, collectively referred to as “the irrigation fluid barrier devices 1510”) and one or more fluid collectors 1522. In the illustrated embodiment, each irrigation fluid barrier device 1510 has a generally elongate, linear form factor and is positioned on either side of a first port 1502 and a second port 1504 to define a reservoir 1520. For example, the first irrigation fluid barrier device 1510a can be positioned closer to the patient's head, the second irrigation fluid barrier device 1510b can be positioned closer to the patient's feet, and each irrigation fluid barrier device 1510 can extend across the patient's width. The irrigation fluid barrier devices 1510 can have a construction similar to the other embodiments of irrigation fluid barrier devices described herein.

The fluid collectors 1522 can be shaped to collect fluid from the reservoir 1520. For example, the fluid collectors 1522 can be bags, sheets, funnels, and/or the like made of a fluid impermeable material and positioned on either side of the patient 101. The irrigation fluid barrier devices 1510 can be shaped, sized, and positioned to direct expelled fluid pooling in the reservoir toward either fluid collector 1522. In some embodiments, the fluid collectors 1522 are each coupled to a corresponding tube 1542 that can direct fluid away from the fluid collectors 1522 toward, for example, a drain. While illustrated schematically in the top view of FIG. 15, the tubes 1542 can be coupled to a bottom portion of the fluid collectors 1522 such that the fluid drains into the tubes 1542. In some embodiments, the tubes 1542 are further coupled to a pump or suction device that forces fluid flow away from the fluid collectors 1522.

Unlike some of the other embodiments illustrated and described herein, the system 1500 includes multiple irrigation fluid barrier devices 1510 that can be independently positioned on the patient 101 as desired. For example, a physician can space the two irrigation fluid barrier devices 1510 farther apart from one another if they wish to define a larger reservoir 1520. Therefore, the system 1500 is an example of a modular multi-portal surgical system that can be customized to the specific patient 101. Also, the illustrated embodiment of FIG. 15 is merely one representative example, and in other embodiments, the system 1500 can include a different number of irrigation fluid barrier devices 1510 (e.g., three, four, or more), irrigation fluid barrier devices 1510 having different shapes (e.g., curved, C-shaped), and/or the like.

It will be appreciated that FIGS. 2-15 merely illustrate example embodiments of an irrigation fluid barrier device configured in accordance with embodiments of the present technology. For example, an irrigation fluid barrier device can have other shapes (e.g., a half-circle arc forming a U shape, a circle, an oval, a triangle, a pentagon, a hexagon, etc.), channel configurations, and/or dimensions.

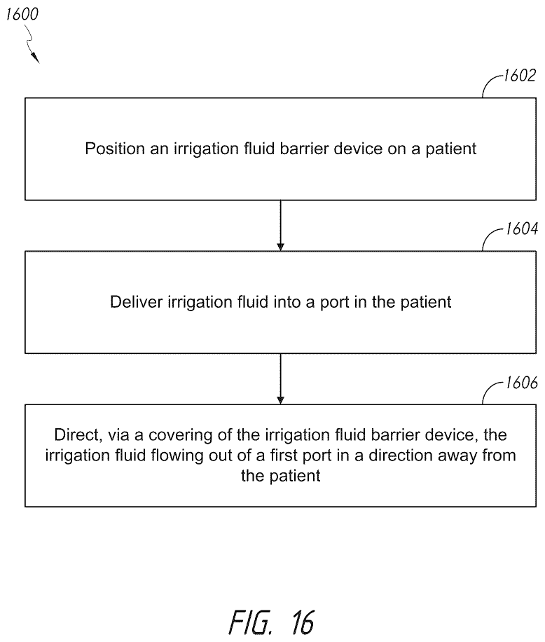

FIG. 16 is a flowchart illustrating a method 1600 for irrigating a surgical site in a patient in accordance with embodiments of the present technology. While the steps of the method 1600 are described below in a particular order, one or more of the steps can be performed in a different order or omitted, and the method 1600 can include additional and/or alternative steps. Additionally, although the method 1600 may be described below with reference to the embodiments of the present technology described herein, the method 1600 can be performed with other embodiments of the present technology.

The method 1600 begins at block 1602 by positioning an irrigation fluid barrier device (e.g., the irrigation fluid barrier device 163, 210, 310) on the patient. The irrigation fluid barrier device can include a core material (e.g., the core material 230) and a covering (e.g., the covering 232) enclosing the core material. The core material and the covering can be shaped to at least partially surround a port (e.g., ports 182 and/or 184) in the patient.

At block 1604, the method 1600 continues by delivering irrigation fluid into a port (e.g., the ports 182 and/or 184) in the patient. The second port can be spaced apart from and in fluid communication with the first port. The irrigation fluid can be delivered using an instrument (e.g., an instrument dedicated to providing irrigation, an endoscope with irrigation lumens, etc.).

At block 1606, the method 1600 continues by directing, via the covering of the irrigation fluid barrier device, the irrigation fluid flowing out of the first port in a direction away from the patient. In some embodiments, the irrigation fluid barrier device is U-shaped (e.g., as shown in FIG. 2) such that the irrigation fluid is directed out through the open end of the U shape. In some embodiments, the irrigation fluid barrier device includes an extension segment (e.g., the out-flow conduit 319) defining a channel (e.g., the passageway 322) therein such that the irrigation fluid is directed out through the channel.

In some embodiments, the method 1600 can further include applying an incise drape over the irrigation fluid barrier device. The incise drape can be configured to stick to the patient and thereby secure a position of the irrigation fluid barrier device relative to the patient. In some embodiments, the method 1600 can further include providing a collector, and the irrigation fluid flowing out of the first port can be directed toward the collector.

### Examples

The present technology is illustrated, for example, according to various aspects described below as numbered examples (1, 2, 3, etc.) for convenience. These are provided as examples and do not limit the present technology. It is noted that any of the dependent examples may be combined in any combination, and placed into a respective independent example. The other examples can be presented in a similar manner.
1. An irrigation fluid barrier device positionable on a back of a patient, the device comprising:
a flexible reservoir body having a flat bottom configured to be placed along the patient, wherein the flexible reservoir body is configured to be coupled to the patient's skin such that the flexible reservoir body is positioned adjacent to a port in the patient and defines (i) a reservoir on the patient to collect irrigation fluid flowing out of the port and (ii) at least one exit opening through which the irrigation fluid flows to exit the reservoir.
2. The device of example 1, wherein the at least one exit opening is positioned to allow the irrigation fluid to exit the reservoir prior to overflowing of the reservoir.
3. The device of example 1 or example 2, further comprising an out-flow conduit connected to the flexible reservoir body, wherein the out-flow conduit defines a passageway in fluid communication with the at least one exit opening.
4. The device of example 3, wherein the passageway includes an open channel.
5. The device of any of examples 1-4, wherein the flexible reservoir body has an elongate body having a first end and an opposing second end, wherein the at least one exit opening is a gap between the first end and the second end.
6. The device of any of examples 1-5, wherein the flexible reservoir body is configured with sufficient rigidity to prevent collapse of the flexible reservoir body when the reservoir is at least partially filled with saline.
7. The device of any of examples 1-6, wherein the flexible reservoir body includes:
a core material; and
a covering enclosing the core material,
wherein the core material and the covering are shaped to, when the device is positioned on the back of the patient, at least partially surround the port in the patient, and
wherein the covering is configured to direct the irrigation fluid flowing out of the port in a direction away from the patient.
8. The device of example 7, wherein the core material and the covering are shaped to, when the device is positioned on the back of the patient, fully surround the port in the patient.
9. The device of example 7 or example 8, wherein the core material includes at least one of a gel or a foam.
10. The device of any of examples 1-9, further comprising an adhesive layer configured to attach the flexible reservoir body to the patient's skin.
11. The device of any of examples 1-10, wherein the device is U-shaped.
12. The device of any of examples 1-10, wherein the device has a rectangular, circular, or ovoid shape.
13. The device of any of examples 1-12, wherein the port is a first port, wherein the flexible reservoir body, when the device is positioned on the back of the patient, at least partially surrounds the first port and a second port in the patient, wherein the second port is spaced apart from and in fluid communication with the first port, and wherein the second port is configured to allow the irrigation fluid to flow into the patient.
14. The device of any of examples 1-13, wherein the device is configured to support a user holding a surgical instrument in the patient.
15. The device of any of example 1-14, further comprising one or more support bases configured to removably couple to the flexible reservoir body and interface the patient.
16. A surgical system, comprising:
an irrigation fluid barrier device including:
   a core material, and
   a covering enclosing the core material,
   wherein the core material and the covering are shaped to, when the irrigation fluid barrier device is positioned on a patient, at least partially surround a port in the patient; and
a flow tube coupleable to the irrigation fluid barrier device, wherein the flow tube is configured to direct irrigation fluid flowing out of the port away from the patient.
17. The surgical system of example 16, further comprising an incise drape configured to be positioned over the irrigation fluid barrier device and stick to the patient, thereby securing a position of the irrigation fluid barrier device relative to the patient.
18. The surgical system of example 16 or example 17, wherein the port is a first port, wherein the core material and the covering are shaped to, when the device is positioned on the patient, at least partially surround the first port and a second port in the patient, and wherein the surgical system further comprises a surgical instrument positionable in the second port and configured to deliver the irrigation fluid to the patient via the second port.
19. A method for irrigating a surgical site in a patient, the method comprising:
positioning an irrigation fluid barrier device on the patient, wherein the irrigation fluid barrier device includes a core material and a covering enclosing the core material, and wherein the core material and the covering are shaped to at least partially surround a first port in the patient;
delivering irrigation fluid into a second port in the patient, wherein the second port is spaced apart from and in fluid communication with the first port; and
directing, via the covering of the irrigation fluid barrier device, the irrigation fluid flowing out of the first port in a direction away from the patient.
20. The method of example 19, further comprising applying an incise drape over the irrigation fluid barrier device, wherein the incise drape is configured to stick to the patient and thereby secure a position of the irrigation fluid barrier device relative to the patient.
21. The method of example 19 or example 20, further comprising providing a collector, wherein directing comprises directing the irrigation fluid flowing out of the first port toward the collector.
22. The method of any of examples 19-21, wherein the irrigation fluid barrier device is U-shaped.
23. The method of any of examples 19-22, wherein the core material includes an extension segment defining a channel therein, and wherein directing comprises directing the irrigation fluid flowing out of the first port through the channel.
24. The method of any of examples 19-23, further comprising:
selecting one or more support bases among a plurality of support bases; and
attaching the selected one or more support bases to the core material and/or the covering in a desired arrangement,
wherein positioning the irrigation fluid barrier device on the patient comprises placing the one or more support bases on the patient according to the desired arrangement.

### Conclusion

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Features from various systems, methods, and instruments can be combined with features disclosed in U.S. application Ser. No. 15/793,950; U.S. application Ser. No. 17/902,685; U.S. application Ser. No. 18/335,737; U.S. application Ser. No. 18/464,949; U.S. application Ser. No. 18/470,140; U.S. application Ser. No. 18/596,610; U.S. application Ser. No. 18/674,784; U.S. Pat. No. 8,632,594; U.S. Pat. No. 9,308,099; U.S. Pat. No. 10,105,238; U.S. Pat. No. 10,201,431; U.S. Pat. No. 10,898,340; U.S. Pat. No. 11,464,648; U.S. Pat. No. 11,950,770; U.S. Pat. No. 9,820,788; U.S. Pat. No. 10,799,367; U.S. Pat. No. 10,322,009; PCT App. No. PCT/US 20/49982; PCT App. No. PCT/US 22/21193; PCT App. No. PCT/US21/63881; PCT App. No. PCT/US22/19706; PCT App. No. PCT/US22/21193; PCT App. No. PCT/US24/18567; PCT App. No. PCT/US23/81937; PCT App. No. PCT/US12/58968; and PCT App. No. PCT/US15/43109, which are hereby incorporated by reference and made a part of this application. This application is related to U.S. Provisional Application No. 63/719,049, filed on Nov. 11, 2024, the disclosure of which is hereby incorporated by reference in its entirety and made a part of this application. All patents and patent applications referenced herein are incorporated by reference in their entireties. Variations of the implants are contemplated.

Systems, components, and instruments disclosed herein can be disposable or reusable. For example, the irrigation fluid barrier devices, instruments, and/or cannulas can be disposable to prevent cross-contamination. As used herein, the term “disposable” when applied to a system or component (or combination of components), such as an instrument, a tool, or a distal tip or a head, is a broad term and generally means, without limitation, that the system or component in question is used a finite number of times and is then discarded. Some disposable components are used only once and are then discarded. In other embodiments, the components and instruments are non-disposable and can be used any number of times. For example, the irrigation fluid barrier devices can be sterilized between uses. Various systems, methods, and techniques described above provide a number of ways to carry out the invention. Of course, it is to be understood that not necessarily all objectives or advantages described may be achieved in accordance with any particular embodiment described herein and may depend on the procedures to be performed, the irrigation fluid barrier device, etc.

Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the present technology. Accordingly, the disclosure and associated technology can encompass other embodiments not expressly shown or described herein.

What is claimed is:

1. An irrigation fluid barrier device positionable on a back of a patient, the device comprising:
a flexible reservoir body having a flat bottom configured to be placed along the patient, wherein the flexible reservoir body is configured to be coupled to the patient's skin such that the flexible reservoir body is positioned adjacent to a port in the patient and defines (i) a reservoir on the patient to collect irrigation fluid flowing out of the port and (ii) at least one exit opening through which the irrigation fluid flows to exit the reservoir.

2. The device of claim 1, wherein the at least one exit opening is positioned to allow the irrigation fluid to exit the reservoir prior to overflowing of the reservoir.

3. The device of claim 1, further comprising an out-flow conduit connected to the flexible reservoir body, wherein the out-flow conduit defines a passageway in fluid communication with the at least one exit opening.

4. The device of claim 3, wherein the passageway includes an open channel.

5. The device of claim 1, wherein the flexible reservoir body has an elongate body having a first end and an opposing second end, wherein the at least one exit opening is a gap between the first end and the second end.

6. The device of claim 1, wherein the flexible reservoir body is configured with sufficient rigidity to prevent collapse of the flexible reservoir body when the reservoir is at least partially filled with saline.

7. The device of claim 1, wherein the flexible reservoir body includes:
a core material; and
a covering enclosing the core material,
wherein the core material and the covering are shaped to, when the device is positioned on the back of the patient, at least partially surround the port in the patient, and
wherein the covering is configured to direct the irrigation fluid flowing out of the port in a direction away from the patient.

8. The device of claim 7, wherein the core material and the covering are shaped to, when the device is positioned on the back of the patient, fully surround the port in the patient.

9. The device of claim 7, wherein the core material includes at least one of a gel or a foam.

10. The device of claim 1, further comprising one or more support bases configured to removably couple to the flexible reservoir body and interface the patient.

11. The device of claim 1, further comprising an adhesive layer configured to attach the flexible reservoir body to the patient's skin.

12. The device of claim 1, wherein the device is U-shaped.

13. The device of claim 1, wherein the device has a rectangular, circular, or ovoid shape.

14. The device of claim 1, wherein the port is a first port, wherein the flexible reservoir body, when the device is positioned on the back of the patient, at least partially surrounds the first port and a second port in the patient, wherein the second port is spaced apart from and in fluid communication with the first port, and wherein the second port is configured to allow the irrigation fluid to flow into the patient.

15. The device of claim 1, wherein the device is configured to support a user holding a surgical instrument in the patient.

16. A surgical system, comprising:
an irrigation fluid barrier device including:
a core material, and
a covering enclosing the core material,
wherein the core material and the covering are shaped to, when the irrigation fluid barrier device is positioned on a patient, at least partially surround a port in the patient; and
a flow tube coupleable to the irrigation fluid barrier device, wherein the flow tube is configured to direct irrigation fluid flowing out of the port away from the patient.

17. The surgical system of claim 16, further comprising an incise drape configured to be positioned over the irrigation fluid barrier device and stick to the patient, thereby securing a position of the irrigation fluid barrier device relative to the patient.

18. The surgical system of claim 16, wherein the port is a first port, wherein the core material and the covering are shaped to, when the device is positioned on the patient, at least partially surround the first port and a second port in the patient, and wherein the surgical system further comprises a surgical instrument positionable in the second port and configured to deliver the irrigation fluid to the patient via the second port.

19. A method for irrigating a surgical site in a patient, the method comprising:
positioning an irrigation fluid barrier device on the patient, wherein the irrigation fluid barrier device includes a core material and a covering enclosing the core material, and wherein the core material and the covering are shaped to at least partially surround a first port in the patient;
delivering irrigation fluid into a second port in the patient, wherein the second port is spaced apart from and in fluid communication with the first port; and
directing, via the covering of the irrigation fluid barrier device, the irrigation fluid flowing out of the first port in a direction away from the patient.

20. The method of claim 19, further comprising applying an incise drape over the irrigation fluid barrier device, wherein the incise drape is configured to stick to the patient and thereby secure a position of the irrigation fluid barrier device relative to the patient.

21. The method of claim 19, further comprising providing a collector, wherein directing comprises directing the irrigation fluid flowing out of the first port toward the collector.

22. The method of claim 19, wherein the irrigation fluid barrier device is U-shaped.

23. The method of claim 19, wherein the core material includes an extension segment defining a channel therein, and wherein directing comprises directing the irrigation fluid flowing out of the first port through the channel.

24. The method of claim 19, further comprising:
selecting one or more support bases among a plurality of support bases; and
attaching the selected one or more support bases to the core material and/or the covering in a desired arrangement,
wherein positioning the irrigation fluid barrier device on the patient comprises placing the one or more support bases on the patient according to the desired arrangement.
